(19) United States
(12) Patent Application Publication
(10) Pub. No.: US 2024/0221903 A1
(43) Pub. Date: Jul. 4, 2024

(54) INFORMATION PROCESSING DEVICE AND NON-TRANSITORY COMPUTER-READABLE STORAGE MEDIUM STORING PROGRAM

(71) Applicant: SEIKO EPSON CORPORATION, Tokyo (JP)

(72) Inventor: Shinichi Kobayashi, Chino-shi (JP)

(73) Assignee: SEIKO EPSON CORPORATION, Tokyo (JP)

(21) Appl. No.: 18/397,005

(22) Filed: Dec. 27, 2023

(30) Foreign Application Priority Data
Dec. 28, 2022 (JP) 2022-211856

(51) Int. Cl. G16H 20/60; G16H 10/20; G16H 15/00

(52) U.S. Cl. CPC G16H 20/60; G16H 10/20; G16H 15/00

(57) ABSTRACT

An information processing device includes a personality information acquisition unit configured to acquire personality information related to a personality of a target person receiving health guidance, a health information acquisition unit configured to acquire health information including a result of a medical examination of the target person, a goal setting unit configured to set a weight loss goal of the target person based on the health information, a storage unit configured to store a trained model in advance, the trained model being configured to output, among a plurality of advice candidates based on the personality information, an advice candidate expected to be valid to achieve the weight loss goal as a guidance content for the target person when the personality information and the weight loss goal are input, and a report generation unit configured to generate a guidance report including the guidance content output from the trained model.

The present application is based on, and claims priority from JP Application Serial Number 2022-211856, filed Dec. 28, 2022, the disclosure of which is hereby incorporated by reference herein in its entirety.

### BACKGROUND

### 1. Technical Field

The present disclosure relates to an information processing device and a non-transitory computer-readable storage medium storing a program.

### 2. Related Art

JP-A-2012-64065 discloses a health guidance support plan generation system that enables a health guidance target person to continuously receive health guidance and can improve a health guidance achievement.

In the technique disclosed in JP-A-2012-64065, guidance on an activity of a target person of specific health guidance is given without reflecting the personality of the target person, which may lower the motivation of the target person for the activity and makes it difficult for the target person to continuously perform the activity based on the guidance content from a counselor.

### SUMMARY

An information processing device according to an aspect of the present disclosure includes a personality information acquisition unit configured to acquire personality information related to a personality of a target person receiving health guidance, a health information acquisition unit configured to acquire health information including a result of a medical examination of the target person, a goal setting unit configured to set a weight loss goal of the target person based on the health information, a storage unit configured to store a trained model in advance, the trained model being configured to output, among a plurality of advice candidates based on the personality information, an advice candidate expected to be valid to achieve the weight loss goal as a guidance content for the target person when the personality information and the weight loss goal are input, and a report generation unit configured to input the personality information and the weight loss goal to the trained model and generate a guidance report including the guidance content output from the trained model.

A non-transitory computer-readable storage medium storing a program according to an aspect of the present disclosure, the program being configured to cause a computer to execute acquiring personality information related to a personality of a target person receiving health guidance, acquiring health information including a result of a medical examination of the target person, setting a weight loss goal of the target person based on the health information, reading a trained model from a storage unit, the trained model being configured to output, among a plurality of advice candidates based on the personality information, an advice candidate expected to be valid to achieve the weight loss goal as a guidance content for the target person when the personality information and the weight loss goal are input, and inputting the personality information and the weight loss goal to the trained model and generating a guidance report including the guidance content output from the trained model.

### BRIEF DESCRIPTION OF THE DRAWING

FIG. 1 is a block diagram illustrating a schematic configuration of an information processing device according to an embodiment.

FIG. 2 is a diagram illustrating examples of a personality pattern derived from the motivation, the strength of will, and the assertiveness.

FIG. 3 is a diagram illustrating examples of a weight loss goal set for a target person.

FIG. 4 is a first diagram illustrating a procedure for generating a trained model.

FIG. 5 is a second diagram illustrating a procedure for generating a trained model.

FIG. 6 is a flowchart illustrating guidance report generation processing.

FIG. 7 illustrates the Johari window.

### DESCRIPTION OF EMBODIMENTS

An embodiment of the present disclosure will be described below with reference to the drawings. Here, in each of the figures below, the sizes of the respective members may be different from the actual sizes in order to make each member recognizable.

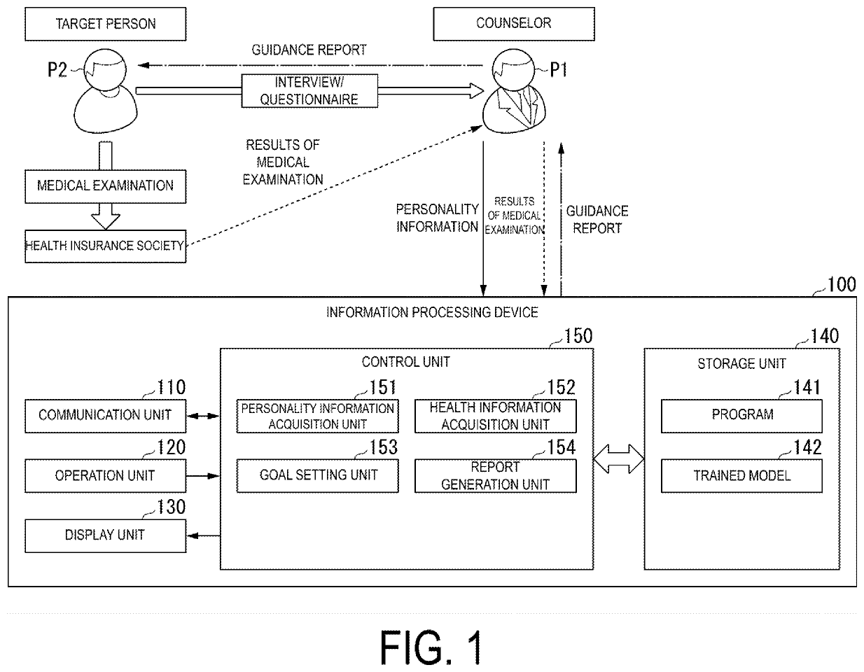

FIG. 1 is a block diagram illustrating a schematic configuration of an information processing device 100 according to an embodiment. The information processing device 100 is a device for supporting specific health guidance performed by a counselor P1 on a target person P2. For example, the information processing device 100 is a personal computer (PC).

For example, the target person P2 is a person who has been diagnosed with metabolic syndrome through a medical examination periodically performed. When the diagnostic result of a waist circumference and at least two diagnostic results of a neutral fat, a fasting blood glucose level, and a blood pressure (systolic blood pressure and diastolic blood pressure) among results of the medical examination correspond to diagnostic criterion values of metabolic syndrome, the person who has undergone the medical examination is diagnosed with metabolic syndrome and becomes a target person of the specific health guidance. In this specification, the specific health guidance may be abbreviated as health guidance.

For example, the diagnostic criterion values of metabolic syndrome related to the waist circumference, the neutral fat, the fasting blood glucose level, and the blood pressure are as follows.

Diagnostic criterion values of metabolic syndrome
(a) Waist circumference: 85 cm or more for men and 90 cm or more for women
(b) Neutral fat: 150 mg/dl or more
(c) Fasting blood glucose level: 110 mg/dl or more
(d) Blood pressure: 130 mmHg or more for a systolic blood pressure and 85 mmHg or more for a diastolic blood pressure

The results of the medical examination of the target person P2 are provided from a health insurance society to the counselor P1. A method of providing the results of the medical examination to the counselor P1 is not particularly limited. For example, the target person P2 himself/herself may provide the counselor P1 with printed matter on which the results of the medical examination are printed. Alternatively, the target person P2 may transmit an E-mail attached with an electronic file of the results of the medical examination to a smartphone, a tablet terminal, or the like that can be used by the counselor P1. In either method, the results of the medical examination of the target person P2 are provided to the counselor P1 in a state of being associated with identification information of the target person P2. For example, the identification information of the target person P2 includes the insurer number of a health insurance society in which the target person P2 is enrolled, the insured number of the target person P2, and the like.

Upon receiving the results of the medical examination of the target person P2 from the health insurance society, the counselor P1 contacts the target person P2 and determines the date and time of a medical interview. The target person P2 has a medical interview with the counselor P1 at the date and time of the medical interview and answers questions from the counselor P1. The counselor P1 acquires personality information of the target person P2 based on answers and reactions of the target person P2 to the questions. For example, the counselor P1 acquires the whether the target person is motivated, the strength of will, and the assertiveness as the personality information of the target person P2.

For example, the counselor P1 determines the whether the target person P2 is motivated based on the attitude of the target person P2 toward an approach of the health guidance and the sense of urgency of the target person P2. For example, the counselor P1 determines that the target person P2 is motivated when the target person P2 falls under at least one of a case where the target person P2 shows a positive attitude to an improvement approach, a case where the target person P2 collects information related to health by himself/herself, a case where the target person P2 has a sense of urgency, or the like. On the other hand, the counselor P1 determines that the target person P2 is not motivated when the target person P2 falls under at least one of a case where the target person P2 shows a negative attitude to the improvement approach (reluctantly participates), a case where the target person P2 does not have a sense of urgency, or the like.

For example, the counselor P1 determines the strength of will of the target person P2 by determining whether the target person P2 can continue effort. For example, the counselor P1 determines that the target person P2 has a strong will when the target person P2 falls under at least one of a case where the target person P2 is hard on himself/herself, a case where the target person P2 has already continuously performed some kind of activity, or the like. On the other hand, the counselor P1 determines that the target person P2 has a weak will when the target person P2 falls under at least one of a case where the target person P2 is lenient to himself/herself, a case where the target person P2 easily makes excuses, a case where the target person P2 mostly cannot stick with anything, or the like.

For example, the counselor P1 determines the assertiveness of the target person P2 based on the attitude of the target person P2 listening to the counselor P1 and responses to questions. For example, the counselor P1 determines that the target person P2 has a weak self-assertiveness when the target person P2 falls under at least one of a case where the target person P2 asks for advice, a case where the target person P2 meekly accepts opinions from the counselor P1, or the like. On the other hand, the counselor P1 determines that the target person P2 has a strong self-assertiveness when the target person P2 falls under at least one of a case where the target person P2 dislikes changing his/her opinions, a case where the target person P2 makes many counterarguments to opinions from the counselor P1, or the like.

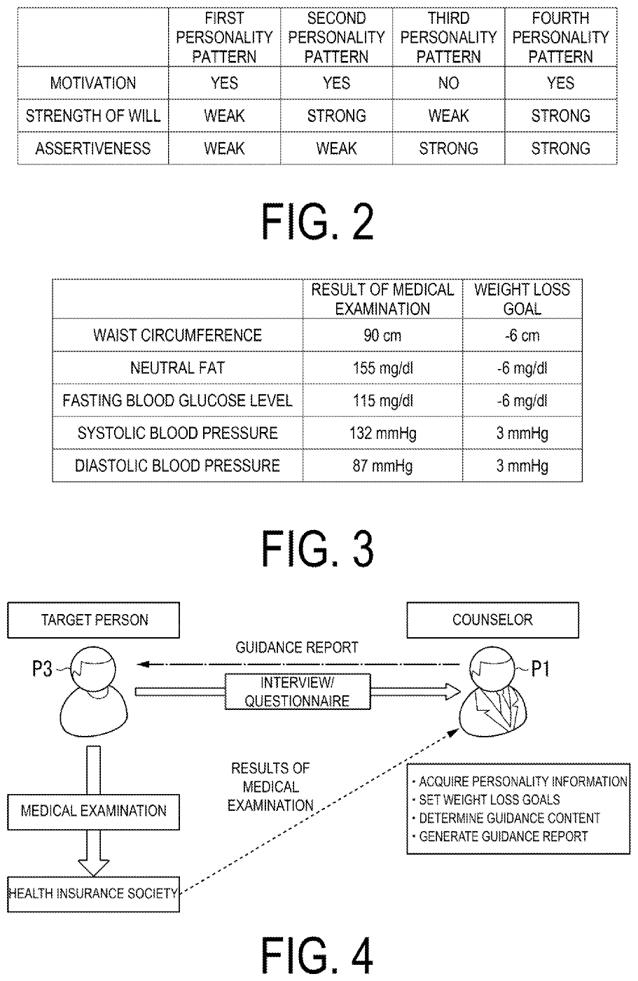

FIG. 2 is a diagram illustrating examples of a personality pattern derived from the whether the target person is motivated, the strength of will, and the assertiveness. As illustrated in FIG. 2, a personality pattern of a person who is motivated, has a weak will, and has a weak self-assertiveness is defined as a first personality pattern. A person image of the target person P2 whose personality pattern derived from the whether the target person is motivated, the strength of will, and the assertiveness corresponds to the first personality pattern is as follows.

Person image of the target person P2 corresponding to the first personality pattern
Showing a positive attitude to the approach and being motivated.
Having a sense of urgency.
Easily stopping the approach because being lenient to himself/herself and making excuses.
Having little knowledge.
Needing advice from others and meekly accepting opinions from the counselor P1.
Doing nothing because not knowing what to do.
Being aware of a problem but unable to take action.

As illustrated in FIG. 2, a personality pattern of a person who is motivated, has a strong will, and has a weak self-assertiveness is defined as a second personality pattern. A person image of the target person P2 whose personality pattern derived from the whether the target person is motivated, the strength of will, and the assertiveness corresponds to the second personality pattern is as follows.

Person image of the target person P2 corresponding to the second personality pattern
Showing a positive attitude to the approach and being motivated.
Having a sense of urgency.
Already engaging in some kind of exercise and measuring the weight at regular intervals.
Having little knowledge.
Needing advice from others and meekly accepting opinions from the counselor P1.
Having awareness of dietary control.

As illustrated in FIG. 2, a personality pattern of a person who is not motivated, has a weak will, and has a strong self-assertiveness is defined as a third personality pattern. A person image of the target person P2 whose personality pattern derived from the whether the target person is motivated, the strength of will, and the assertiveness corresponds to the third personality pattern is as follows.
Person image of the target person P2 corresponding to the third personality pattern
Showing a negative attitude to the approach and reluctantly participating.
Having no sense of urgency.
Easily stopping the approach because being lenient to himself/herself and making excuses.
Having little knowledge.
Making many counterarguments to opinions from the counselor P1.

As illustrated in FIG. 2, a personality pattern of a person who is motivated, has a strong will, and has a strong self-assertiveness is defined as a fourth personality pattern. A person image of the target person P2 whose personality pattern derived from the whether the target person is motivated, the strength of will, and the assertiveness corresponds to the fourth personality pattern is as follows.

Person image of the target person P2 corresponding to the fourth personality pattern
Showing a positive attitude to the approach and being motivated.
Having a sense of urgency.
Already engaging in some kind of exercise and measuring the weight at regular intervals.
Having much knowledge.
Having confidence in that what he/she is doing is correct and making many counterarguments to opinions from the counselor P1.

Returning to FIG. 1, the description will be continued below. As described above, the counselor P1 acquires the whether the target person is motivated, the strength of will, and the assertiveness as the personality information of the target person P2 and then operates the information processing device 100 to input the identification information, the personality information, and the results of the medical examination of the target person P2 to the information processing device 100. The results of the medical examination input to the information processing device 100 are the results of the medical examination when the target person P2 has been diagnosed with metabolic syndrome.

The information processing device 100 generates a guidance report including a guidance content for the target person P2 based on the personality information and the results of the medical examination of the target person P2, which are input by the counselor P1. The information processing device 100 provides the generated guidance report to the counselor P1. A method of providing the guidance report to the counselor P1 is not particularly limited. For example, when a printer is coupled to the information processing device 100, the information processing device 100 may provide printed matter on which the content of the guidance report is printed to the counselor P1 via the printer. Alternatively, the information processing device 100 may transmit an E-mail attached with an electronic file of the guidance report to a smartphone, a tablet terminal, or the like that can be used by the counselor P1.

After checking the content of the guidance report, the counselor P1 provides the guidance report to the target person P2. A method of providing the guidance report to the target person P2 is not particularly limited. For example, when the counselor P1 obtains the guidance report as printed matter, the printed matter of the guidance report may be provided to the target person P2. Alternatively, when the counselor P1 obtains the guidance report as an electronic file, an E-mail attached with the electronic file of the guidance report may be transmitted to a user terminal of the target person P2. Examples of the user terminal of the target person P2 include a smartphone and a tablet terminal that can be used by the target person P2.

Hereinafter, the configuration of the information processing device 100 will be described in detail. As illustrated in FIG. 1, the information processing device 100 includes a communication unit 110, an operation unit 120, a display unit 130, a storage unit 140, and a control unit 150.

The communication unit 110 is a communication interface device communicating with an external device. For example, when the E-mail attached with the electronic file of the guidance report is provided to the counselor P1, the communication unit 110 transmits the E-mail attached with the electronic file of the guidance report to the smartphone, the tablet terminal, or the like that can be used by the counselor P1 in accordance with an instruction from the control unit 150.

The operation unit 120 is a device receiving an input operation from the counselor P1 to the information processing device 100. Examples of the operation unit 120 include an input device such as a keyboard and a mouse. The operation unit 120 outputs, as an operation signal, an electric signal generated when the keyboard, the mouse, or the like is operated to the control unit 150. Upon receiving an operation of inputting the identification information, the personality information, and the results of the medical examination of the target person P2 from the counselor P1, the operation unit 120 outputs an electric signal generated by the input operation to the control unit 150 as an operation signal.

The display unit 130 displays a predetermined image by being controlled by the control unit 150. Examples of the display unit 130 include a liquid crystal monitor and an organic electro luminescence (EL) monitor.

The storage unit 140 stores a program, data, and the like necessary for the control unit 150 to perform various types of processing. For example, the storage unit 140 includes a nonvolatile storage device such as a hard disk drive (HDD), a flash memory, and a read only memory (ROM), and a volatile storage device such as a random access memory (RAM).

The storage unit 140 stores a program 141, a trained model 142, and the like. The program 141 includes, in addition to an operating system (OS) and an application program, a program for causing the control unit 150 to execute guidance report generation processing described below, and the like. In the following description, the program for causing the control unit 150 to execute the guidance report generation processing is referred to as a guidance report generation program. The trained model 142 will be described in detail below.

The control unit 150 controls overall operations of the information processing device 100 in accordance with the program 141 stored in the storage unit 140. Examples of the control unit 150 include a processor such as a central processing unit (CPU). The control unit 150 includes a personality information acquisition unit 151, a health information acquisition unit 152, a goal setting unit 153, and a report generation unit 154. These constitutional units are functions implemented by the control unit 150 executing the above guidance report generation program.

Some or all of the functions of the control unit 150 may be implemented by hardware such as a large scale integration (LSI), an application specific integrated circuit (ASIC), a field-programmable gate array (FPGA), or a graphics processing unit (GPU), or may be implemented by cooperation of software and hardware.

The personality information acquisition unit 151 acquires personality information related to the personality of the target person P2 based on the operation signal output from the operation unit 120. For example, the personality information acquisition unit 151 acquires the whether the target person is motivated, the strength of will, and the assertiveness as the personality information of the target person P2.

The health information acquisition unit 152 acquires health information including the results of the medical examination of the target person P2 based on the operation signal output from the operation unit 120. For example, the health information includes, as the results of the medical examination, diagnostic results such as the weight, the chest circumference, the waist circumference, the neutral fat, the fasting blood glucose level, the blood pressure (systolic blood pressure and diastolic blood pressure), and the body mass index (BMI) of the target person P2. These diagnostic items are mere examples, and the results of the medical examination may include diagnostic results of other diagnostic items.

The goal setting unit 153 sets a weight loss goal of the target person P2 based on the health information. For example, the goal setting unit 153 sets weight loss goals such that the diagnostic results of the waist circumference, the neutral fat, the fasting blood glucose level, and the blood pressure of the target person P2 are less than the diagnostic criterion values of metabolic syndrome.

FIG. 3 is a diagram illustrating examples of the weight loss goal set for the target person P2. In the example of FIG. 3, the target person P2 is a 40-year-old male, the diagnostic result of the waist circumference is 90 cm, the diagnostic result of the neutral fat is 155 mg/dl, the diagnostic result of the fasting blood glucose level is 115 mg/dl, the diagnostic result of the systolic blood pressure is 132 mmHg, and the diagnostic result of the diastolic blood pressure is 87 mmHg.

In this case, the goal setting unit 153 sets the weight loss goal of the waist circumference to −6 cm, the weight loss goal of the neutral fat to −6 mg/dl, the weight loss goal of the fasting blood glucose level to −6 mg/dl, the weight loss goal of the systolic blood pressure to −3 mmHg, and the weight loss goal of the diastolic blood pressure to −3 mmHg.

The weight loss goals of the target person P2 set by the goal setting unit 153 may be goals related to the weight and the abdominal circumference of the target person P2. The goal related to the weight of the target person P2 may be set, for example, such that the weight is reduced by substantially several percent per month with respect to the weight of the target person P2 in the medical examination in which the target person P2 has been diagnosed with metabolic syndrome, or may be set such that the weight is reduced to a weight at which the BMI of the target person P2 is 25 or less. By setting the weight loss goal as a goal related to the weight of the target person P2, the target person P2 can easily check the accomplishment of the activity performed by himself/herself, and thus it is easy to maintain a motivation for the activity.

Returning to FIG. 1, the report generation unit 154 inputs the personality information and the weight loss goals to the trained model 142 and generates a guidance report including a guidance content output from the trained model 142. The trained model 142 is a mathematical model that outputs, among a plurality of advice candidates based on the personality information, an advice candidate with which the weight loss goals are estimated to be achieved as the guidance content for the target person P2 when the personality information and the weight loss goals are input. The guidance report may include other information such as the results of the medical examination and the weight loss goals, in addition to the guidance content output from the trained model 142.

Examples of the advice candidate include advice related to exercise. Examples of the advice related to exercise include introduction of exercise performed by the target person P2, advice on exercise performed by the target person P2, and advice on how to walk in daily life. The advice candidate may include not only advice related to exercise as described above but also advice related to meals and sleep. Examples of the advice related to meals include advice related to an amount of each meal, a time when each meal is eaten, and a nutritional balance of each meal. Examples of the advice related to sleep include advice about a wake-up time and a bedtime.

Examples of the plurality of advice candidates based on the personality information include a simple guidance content, an advanced guidance content, a very simple guidance content, and a guidance content respecting the will of the target person P2 as described below.

Simple guidance Content
Guidance related to meals: giving guidance so as to restrict carbohydrate and greasy food and restrict eating between meals.
Guidance related to exercise: guidance on walking, squatting, and how to walk.
Guidance related to sleep: giving guidance so as to have a fixed sleep time.
Tendency of advice: giving advice so as to support continuation.

### Advanced Guidance Content

Guidance related to meals: giving guidance so as to restrict carbohydrate and greasy food more than the simple guidance, encourage meals with more vegetables and protein, and restrict eating between meals.
Guidance related to exercise: guidance on running, squatting, and how to walk.
Guidance related to sleep: giving guidance so as to go to bed at the same time and wake up at the same time.
Tendency of advice: giving advice of encouraging the approach and praising an accomplishment.

### Very Simple Guidance Content

Guidance related to meals: giving guidance so as to record the contents of meals and restrict eating between meals.
Guidance related to exercise: guidance on walking and how to walk.
Guidance related to sleep: giving guidance so as to have a fixed sleep time.
Tendency of advice: giving advice so as to make the target person P2 aware the current problem.

### Guidance Content Respecting the Will of the Target Person P2

Guidance related to meals: recording the contents of meals.
Guidance related to exercise: giving guidance on exercise performed by the target person P2 and describing information about exercise recommended by the counselor P1.
Guidance related to sleep: describing information about guidance related to a sleep time recommended by the counselor P1.
Tendency of advice: giving advice of praising for an activity of the target person P2 and telling an accomplishment (increase or decrease in numerical value) achieved by his/her activity as it is.

For example, the trained model 142 performs learning so as to output the simple guidance content among the advice candidates described above when the personality information representing the first personality pattern is input. The reason why the simple guidance content is adopted in the case of the first personality pattern in this way is that since the target person P2 has a weak will, this guidance is to focus on continuation of the activity and maintain a motivation for the activity by giving advice of praising the continuation.

The reason why the advanced guidance content is not adopted in the case of the first personality pattern is that since the target person P2 is motivated but has a weak will, it is difficult to continue the activity with the advanced guidance. The reason why the very simple guidance content is not adopted in the case of the first personality pattern is that the content is too simple and thus lowers motivation for the activity. The reason why the guidance content respecting the will of the target person is not adopted in the case of the first personality pattern is that the target person P2 has little knowledge and thus cannot take action unless what to do is specifically presented.

For example, the trained model 142 performs learning so as to output the advanced guidance content among the advice candidates when the personality information representing the second personality pattern is input. The reason why the advanced guidance content is adopted in the case of the second personality pattern in this way is that since the target person P2 is motivated and has a strong will, the target person P2 has difficulty in maintaining a motivation with the simple guidance content and cannot continue the activity. This is also because this guidance is to encourage further improvement of a motivation by praising for the activity and the accomplishment by the activity.

The reason why the simple guidance content is not adopted in the case of the second personality pattern is that the content is too simple and thus lowers motivation for the activity. The reason why the very simple guidance content is not adopted in the case of the second personality pattern is that the content is too simple and thus lowers a motivation for the activity. The reason why the guidance content respecting the will of the target person is not adopted in the case of the second personality pattern is that the target person P2 has little knowledge and thus cannot take action unless what to do is specifically presented.

For example, the trained model 142 performs learning so as to output the very simple guidance content among the advice candidates described above when the personality information representing the third personality pattern is input. The reason why the very simple guidance content is adopted in the case of the third personality pattern in this way is that since the target person P2 is not motivated and has a weak will, the goal is to make the target person P2 interested in the activity first. This guidance also allows the target person P2 to understand the current problem and the meaning of the activity being performed.

The reason why the simple guidance content is not adopted in the case of the third personality pattern is that since the target person P2 is not motivated and has a weak will, even a too simple content lowers a motivation for the activity. The reason why the advanced guidance content is not adopted in the case of the third personality pattern is that since the target person P2 is not motivated and has a weak will, it is difficult to continue the activity with the advanced guidance. The reason why the guidance content respecting the will of the target person is not adopted in the case of the third personality pattern is that since the target person P2 has little knowledge and thus cannot take action unless what to do is specifically presented.

For example, the trained model 142 performs learning so as to output the guidance content respecting the will of the target person among the advice candidates when the personality information representing the fourth personality pattern is input. The reason why the guidance content respecting the will of the target person is adopted in the case of the fourth personality pattern is that since forcible guidance of contents about meals, exercise, and the like eliminates a motivation for the activity, this guidance can give an opportunity for the target person P2 to listen to opinions from the counselor P1 even slightly by presenting the desired frequency of the exercise that the target person P2 would like to perform and notifying the result or the accomplishment of the exercise performed by the target person as they are.

The reason why the simple guidance content is not adopted in the case of the fourth personality pattern is that this is to guide the contents of exercise and meals and thus lowers motivation of the target person. The reason why the advanced guidance content is not adopted in the case of the fourth personality pattern is that this is to guide the contents of exercise and meals and thus lowers a motivation of the target person. The reason why the very simple guidance content is not adopted in the case of the fourth personality pattern is that this is to guide the contents of exercise and meals and thus lowers a motivation of the target person.

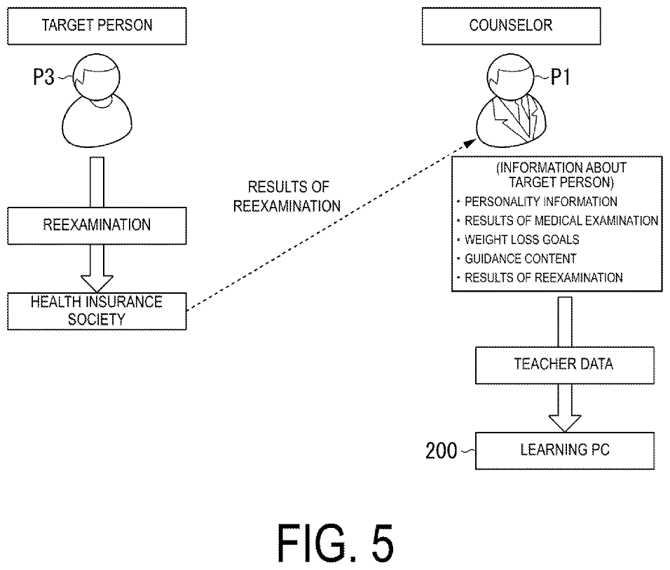

The trained model 142 is a mathematical model generated by machine learning such as deep learning. Hereinafter, a procedure for generating the trained model 142 will be described with reference to FIGS. 4 and 5.

In FIG. 4, a target person P3 is a person who is diagnosed with metabolic syndrome through a medical examination periodically performed and is a person different from the target person P2. The results of the medical examination of the target person P3 are provided from the health insurance society to the counselor P1.

Upon receiving the results of the medical examination of the target person P3 from the health insurance society, the counselor P1 contacts the target person P3 and determines the date and time of a medical interview. The target person P3 makes a medical interview with the counselor P1 at the date and time of the medical interview and answers questions from the counselor P1. The counselor P1 acquires personality information of the target person P3 based on answers and reactions of the target person P3 to the questions. For example, the counselor P1 acquires the whether the target person is motivated, the strength of will, and the assertiveness as the personality information of the target person P3.

In addition, the counselor P1 sets a weight loss goal of the target person P3 based on the results of the medical examination provided from the health insurance society. When goals related to the diagnostic criteria of metabolic syndrome are set, the counselor P1 sets weight loss goals such that the diagnostic results of the waist circumference, the neutral fat, the fasting blood glucose level, and the blood pressure of the target person P3 are less than the diagnostic criterion values of metabolic syndrome.

Then, the counselor P1 determines, based on the personality information of the target person P3, that, among the plurality of advice candidates, an advice candidate for achieving the weight loss goals is a guidance content for the target person P3. For example, when the personality information represents the first personality pattern, the counselor P1 determines that the simple guidance content is the guidance content for the target person P3. When the personality information represents the second personality pattern, the counselor P1 determines that the advanced guidance content is the guidance content for the target person P3. When the personality information represents the third personality pattern, the counselor P1 determines that the very simple guidance content is the guidance content for the target person P3. When the personality information represents the fourth personality pattern, the counselor P1 determines that the guidance content respecting the will of the target person is the guidance content for the target person P3.

The counselor P1 generates a guidance report including the guidance content determined as described above, the results of the medical examination of the target person P3, and the weight loss goals. The counselor P1 provides the generated guidance report to the target person P3.

As illustrated in FIG. 5, the target person P3 undergoes a reexamination after having a daily life according to the guidance report received from the counselor P1. Examples of the reexamination include a medical examination that the target person P3 undergoes after receiving the guidance report. The reexamination may be the same as the medical examination in which the target person P2 has been diagnosed with metabolic syndrome, or may be a simpler examination than the medical examination in which the target person P2 has been diagnosed with metabolic syndrome, for example, an examination of measuring the weight and the abdominal circumference. When the reexamination is the examination of measuring the weight and the abdominal circumference, goals set as the weight loss goals may be goals related to the weight and the abdominal circumference.

When the target person P3 undergoes the reexamination, the results of the reexamination of the target person P3 are provided to the counselor P1. Here, the counselor P1 may receive the results of the reexamination of the target person P3 via the health insurance society or not via the health insurance society.

The counselor P1 checks whether the weight loss goals of the target person P3 have been achieved with the guidance content described in the guidance report based on the results of the reexamination of the target person P3. That is, when the weight loss goals are goals related to the diagnostic criteria of metabolic syndrome, the counselor P1 checks whether the diagnostic results of the waist circumference, the neutral fat, the fasting blood glucose level, and the blood pressure of the target person P3 among the results of the reexamination are less than the diagnostic criterion values of metabolic syndrome.

As described above, the counselor P1 acquires, as information related to the target person P3, the personality information, the results of the medical examination, the weight loss goals, the guidance content, and the results of the reexamination. The results of the reexamination are information indicating whether the weight loss goals of the target person P3 have been achieved with the guidance content described in the guidance report.

As is well known, in supervised learning, which is one of machine learning techniques, by using a set of data to be input (learning data) and data to be output (correct answer data) as teacher data, a correlation between the learning data and the correct answer data is learned. By performing the supervised learning using a large amount of such teacher data, a trained model is generated that outputs correct answer data corresponding to input data when unknown data is input.

Among the information related to the target person P3, the counselor P1 acquires the personality information and the weight loss goals as data to be input, that is, learning data. The counselor P1 also acquires, among the information related to the target person P3, the guidance content with which the weight loss goals of the target person P3 have been able to be achieved as data to be output, that is, correct answer data.

The counselor P1 acquires the set of the learning data and the correct answer data as the teacher data and stores the acquired teacher data in a learning PC 200 in association with the identification information of the target person P3. The learning PC 200 may be the same PC as the information processing device 100 or a different PC from the information processing device 100. When the weight loss goals of the target person P3 have not been able to be achieved, the counselor P1 does not need to acquire the teacher data related to the target person P3.

The same procedure as described above is sequentially applied to target persons other than the target person P3, and thus teacher data associated with identification information of the target persons other than the target person P3 is sequentially stored in the learning PC 200. When the number of pieces of the teacher data stored in the learning PC 200 reaches a predetermined number, the counselor P1 operates the learning PC 200 such that supervised learning is performed based on the stored teacher data.

As a result, a trained model is generated that outputs, as a guidance content for an unknown target person, correct answer data corresponding to input data, that is, an advice candidate with which the weight loss goals of the unknown target person are estimated to be achieved among a plurality of advice candidates when data including the personality information and the weight loss goals of the unknown target person is input.

The trained model generated by the learning PC 200 in this way is the trained model 142 of the embodiment. That is, the trained model 142 is a mathematical model that outputs, among the plurality of advice candidates based on the personality information, the advice candidate with which the weight loss goals of the target person P2, who is an unknown target person, are estimated to be achieved as the guidance content for the target person P2 when the personality information and the weight loss goals of the target person P2 are input.

Next, an operation of the information processing device 100 configured as described above will be described.

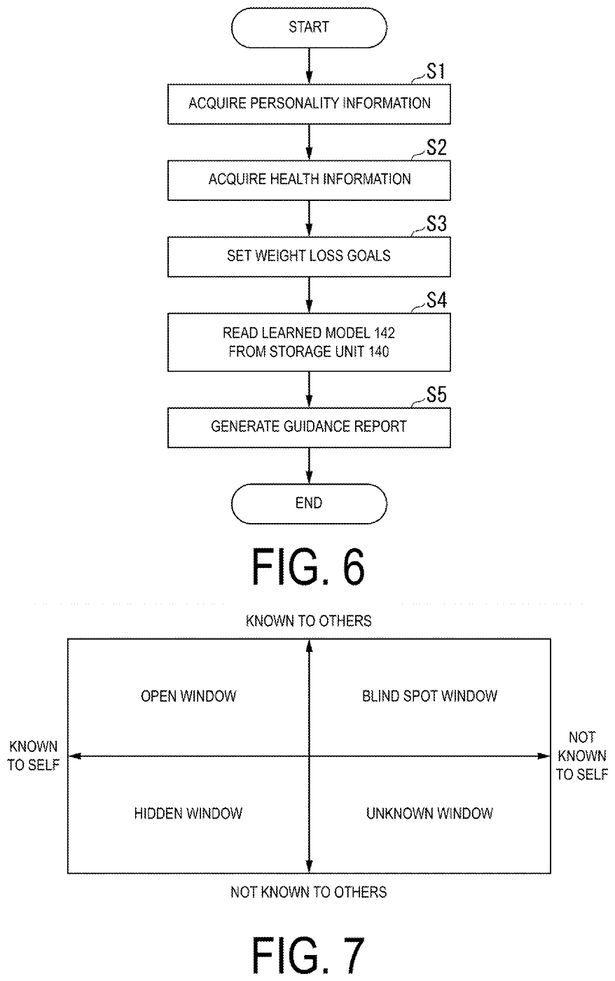

FIG. 6 is a flowchart illustrating the guidance report generation processing performed by the control unit 150. When the control unit 150 detects receipt of an operation for instructing generation of a guidance report from the counselor P1 based on an operation signal output from the operation unit 120, the control unit 150 reads, from the storage unit 140, and executes the guidance report generation program to execute the guidance report generation processing illustrated in FIG. 6.

As illustrated in FIG. 6, when starting the guidance report generation processing, the control unit 150 first acquires personality information related to the personality of the target person P2 based on the operation signal output from the operation unit 120 (step S1). For example, the control unit 150 acquires the whether the target person is motivated, the strength of will, and the assertiveness as the personality information of the target person P2. Since the processing of step S1 is the same as the processing executed by the personality information acquisition unit 151, a detailed description of step S1 is omitted.

Subsequently, the control unit 150 acquires health information including results of a medical examination of the target person P2 based on the operation signal output from the operation unit 120 (step S2). For example, the health information includes, as the results of the medical examination, diagnostic results such as the weight, the chest circumference, the waist circumference, the neutral fat, the fasting blood glucose level, the blood pressure (systolic blood pressure and diastolic blood pressure), and the BMI of the target person P2. Since the processing of step S2 is the same as the processing executed by the health information acquisition unit 152, a detailed description of step S2 is omitted. Note that the execution order of steps S1 and S2 may be reversed. That is, the processing of step S1 may be executed after the processing of step S2.

Subsequently, the control unit 150 sets a weight loss goal of the target person P2 based on the health information (step S3). For example, in step S3, the control unit 150 sets weight loss goals such that the diagnostic results of the waist circumference, the neutral fat, the fasting blood glucose level, and the blood pressure of the target person P2 are less than diagnostic criterion values of metabolic syndrome. Since the processing of step S3 is the same as the processing executed by the goal setting unit 153, a detailed description of step S3 is omitted.

Subsequently, the control unit 150 reads the trained model 142 from the storage unit 140 (step S4). Then, the control unit 150 inputs the personality information and the weight loss goals to the trained model 142 and generates a guidance report including a guidance content output from the trained model 142 (step S5). The control unit 150 may generate a guidance report including other information such as the results of the medical examination and the weight loss goals, in addition to the guidance content output from the trained model 142. Since the processing of step S5 is the same as the processing executed by the report generation unit 154, a detailed description of step S5 is omitted.

### Effects of Embodiment

As described above, the information processing device 100 according to the embodiment includes the personality information acquisition unit 151 configured to acquire personality information related to a personality of the target person P2 receiving health guidance, the health information acquisition unit 152 configured to acquire health information including a result of a medical examination of the target person P2, the goal setting unit 153 configured to set a weight loss goal of the target person P2 based on the health information, the storage unit 140 configured to store the trained model 142 in advance, the trained model 142 being configured to output, among a plurality of advice candidates based on the personality information, an advice candidate expected to be valid to achieve the weight loss goal as a guidance content for the target person P2 when the personality information and the weight loss goal are input, and the report generation unit 154 configured to input the personality information and the weight loss goal to the trained model and generate a guidance report including the guidance content output from the trained model 142.

As described above, the information processing device 100 inputs the personality information and the weight loss goal of the target person P2 to the trained model 142 and generates the guidance report including the guidance content output from the trained model 142. The trained model 142 is a mathematical model generated by machine learning such that among the plurality of advice candidates based on the personality information, the advice candidate expected to be valid to achieve the weight loss goal is output as the guidance content for the target person P2 when the personality information and the weight loss goal of the target person P2 are input. Thus, even when any kind of personality information and weight loss goal are input to the trained model 142, the trained model 142 always outputs a correct guidance content, that is, the guidance content with which the weight loss goal has been achieved in the past achievement.

Thus, according to the information processing device 100 of the embodiment, it is possible to generate the guidance report including the guidance content suitable for the personality of the target person P2 receiving the health guidance, which thus can support the target person P2 to continuously perform the activity based on the guidance content from the counselor P1.

In the embodiment, the personality information acquisition unit 151 acquires, as the personality information of the target person P2, the whether the target person is motivated, the strength of will, and the assertiveness of the target person P2.

According to the embodiment as described above, by using the whether the target person P2 is motivated, the strength of will, and the assertiveness of the target person P2 as the personality information of the target person P2, it is possible to generate the guidance report including the guidance content more suitable for the personality of the target person P2, which thus can more efficiently support the target person P2 to continuously perform the activity based on the guidance content from the counselor P1.

In the embodiment, the health information includes, as the result of the medical examination, diagnostic results of the waist circumference, the neutral fat, the fasting blood glucose level, and the blood pressure of the target person P2. The goal setting unit 153 sets the weight loss goal such that the diagnostic results of the waist circumference, the neutral fat, the fasting blood glucose level, and the blood pressure are less than the diagnostic criterion values of metabolic syndrome.

According to the embodiment, the weight loss goal is set such that the diagnostic results of the waist circumference, the neutral fat, the fasting blood glucose level, and the blood pressure of the target person P2 are less than the diagnostic criterion values of metabolic syndrome. Thus, it is possible to appropriately set the weight loss goal in accordance with the result of the medical examination of the target person P2.

A non-transitory computer-readable storage medium storing the program 141 of the embodiment, the program 141 being configured to cause a computer to execute acquiring personality information related to a personality of the target person P2 receiving health guidance (step S1), acquiring health information including a result of a medical examination of the target person P2 (step S2), setting a weight loss goal of the target person P2 based on the health information (step S3), reading the trained model 142 from the storage unit 140, the trained model being configured to output, among a plurality of advice candidates based on the personality information, an advice candidate expected to be valid to achieve the weight loss goal as a guidance content for the target person P2 when the personality information and the weight loss goal are input (step S4), and inputting the personality information and the weight loss goal to the trained model 142 and generating a guidance report including the guidance content output from the trained model 142 (step S5).

According to the program 141 of the embodiment, it is possible to generate the guidance report including the guidance content suitable for the personality of the target person P2 receiving the health guidance, which thus can support the target person P2 to continuously perform the activity based on the guidance content from the counselor P1.

Although the embodiment of the present disclosure has been described above, the technical scope of the present disclosure is not limited to the above-described embodiment, and various modifications can be made without departing from the gist of the present disclosure.

For example, in the above-described embodiment, a case has been exemplified in which the personality information acquisition unit 151 acquires the whether the target person P2 is motivated, the strength of will, and the assertiveness of the target person P2 as the personality information of the target person P2. The personality information acquisition unit 151 may acquire, as the personality information of the target person P2, a result of a medical interview sheet or a questionnaire used in a medical interview of the target person P2, instead of the whether the target person P2 is motivated, the strength of will, and the assertiveness of the target person P2.

For example, a questionnaire as illustrated in FIG. 2 of JP-A-2004-164087 can be used as a questionnaire for testing the personality of the target person P2. By using the result of the medical interview sheet or the questionnaire used in the medical interview with the target person P2 as the personality information of the target person P2 in this way, it is possible to generate the guidance report including the guidance content more suitable for the personality of the target person P2, which thus can more efficiently support the target person P2 to continuously perform the activity based on the guidance content from the counselor P1.

In addition, the personality information acquisition unit 151 may acquire, as the personality information of the target person P2, a result of a personality analysis using the Johari window performed on the target person P2, instead of the whether the target person P2 is motivated, the strength of will, and the assertiveness of the target person P2. FIG. 7 illustrates the Johari window. As is well known, the Johari window is one psychological model used for self-analysis and is a technique for classifying the characteristics of oneself into four windows by analyzing information about oneself viewed by oneself and information about oneself viewed by others.

As illustrated in FIG. 7, the four windows include an open window, a blind spot window, a hidden window, and an unknown window. The open window indicates oneself known to oneself and others, and one's personality understood by oneself and others corresponds to this window. The blind spot window indicates oneself that cannot be noticed by oneself but is known to others, and one's own habit of thinking or unexpected advantages or disadvantages correspond to this window. The hidden window indicates oneself that is not known to others and is known only to oneself, and a complex, trauma, or the like corresponds to this window. The unknown window indicates oneself not known to oneself or others, and a potential talent or personality corresponds to this window.

For example, the counselor P1 analyzes the personality corresponding to the blind spot window of the target person P2 by utilizing a questionnaire as disclosed in JP-A-2020-166621 and inputs the analysis result of the personality corresponding to the blind spot window to the information processing device 100 as the personality information. Thus, the personality information acquisition unit 151 can acquire the analysis result of the personality corresponding to the blind spot window of the target person P2 as the personality information. As described above, the personality corresponding to the blind spot window is one's personality or the like not noticed by oneself but is known to others. By using the result of the personality analysis using the Johari window as the personality information of the target person P2 in this way, it is possible to generate the guidance report including the guidance content more suitable for the personality of the target person P2, which thus can more efficiently support the target person P2 to continuously perform the activity based on the guidance content from the counselor P1.

### Summary of Present Disclosure

A summary of the present disclosure will be appended below.

### APPENDIX 1

An information processing device including a personality information acquisition unit configured to acquire personality information related to a personality of a target person receiving health guidance, a health information acquisition unit configured to acquire health information including a result of a medical examination of the target person, a goal setting unit configured to set a weight loss goal of the target person based on the health information, a storage unit configured to store a trained model in advance, the trained model being configured to output, among a plurality of advice candidates based on the personality information, an advice candidate expected to be valid to achieve the weight loss goal as a guidance content for the target person when the personality information and the weight loss goal are input, and a report generation unit configured to input the personality information and the weight loss goal to the trained model and generate a guidance report including the guidance content output from the trained model.

As described above, the information processing device according to Appendix 1 inputs the personality information and the weight loss goal of the target person to the trained model and generates the guidance report including the guidance content output from the trained model. The trained model is a mathematical model generated by machine learning such that among the plurality of advice candidates based on the personality information, the advice candidate expected to be valid to achieve the weight loss goal is output as the guidance content for the target person when the personality information and the weight loss goal of the target person are input. Thus, even any kind of personality information and weight loss goal are input to the trained model, the trained model always outputs a correct guidance content, that is, a guidance content with which the weight loss goal has been achieved in the past achievement.

As a result, according to the information processing device described in Appendix 1, it is possible to generate the guidance report including the guidance content suitable for the personality of the target person receiving the health guidance, which thus can support the target person to continuously perform the activity based on the guidance content from the counselor.

### APPENDIX 2

The information processing device according to Appendix 1, wherein the personality information acquisition unit acquires, as the personality information, whether the target person is motivated, a strength of will, and assertiveness of the target person.

According to the information processing device described in Appendix 2, by using the whether the target person is motivated, the strength of will, and the assertiveness of the target person as the personality information of the target person, it is possible to generate the guidance report including the guidance content more suitable for the personality of the target person, which thus can more efficiently support the target person to continuously perform the activity based on the guidance content from the counselor.

### APPENDIX 3

The information processing device according to Appendix 1, wherein the personality information acquisition unit acquires, as the personality information, a result of a medical interview sheet or a questionnaire used in a medical interview of the target person.

According to the information processing device described in Appendix 3, by using the result of the medical interview sheet or the questionnaire used in the medical interview of the target person as the personality information of the target person, it is possible to generate the guidance report including the guidance content more suitable for the personality of the target person, which thus can more efficiently support the target person to continuously perform the activity based on the guidance content from the counselor.

### APPENDIX 4

The information processing device according to Appendix 1, wherein the personality information acquisition unit acquires, as the personality information, a result of a personality analysis performed on the target person by using a Johari window.

According to the information processing device described in Appendix 4, by using the result of the personality analysis using the Johari window as the personality information of the target person, it is possible to generate the guidance report including the guidance content more suitable for the personality of the target person, which thus can more efficiently support the target person to continuously perform the activity based on the guidance content from the counselor.

### APPENDIX 5

The information processing device according to one of Appendixes 1 to 4, wherein the health information includes, as the result of the medical examination, diagnostic results of a waist circumference, a neutral fat, a fasting blood glucose level, and a blood pressure of the target person, and the goal setting unit sets the weight loss goal such that the diagnostic results of the waist circumference, the neutral fat, the fasting blood glucose level, and the blood pressure are less than diagnostic criterion values of metabolic syndrome.

According to the information processing device described in Appendix 5, the weight loss goal is set such that the diagnostic results of the waist circumference, the neutral fat, the fasting blood glucose level, and the blood pressure of the target person are less than the diagnostic criterion values of metabolic syndrome. Thus, the weight loss goal can be appropriately set according to the result of the medical examination of the target person.

### APPENDIX 6

A non-transitory computer-readable storage medium storing a program, the program being configured to cause a computer to execute acquiring personality information related to a personality of a target person receiving health guidance, acquiring health information including a result of a medical examination of the target person, setting a weight loss goal of the target person based on the health information, reading a trained model from a storage unit, the trained model being configured to output, among a plurality of advice candidates based on the personality information, an advice candidate expected to be valid to achieve the weight loss goal as a guidance content for the target person when the personality information and the weight loss goal are input, and inputting the personality information and the weight loss goal to the trained model and generating a guidance report including the guidance content output from the trained model.

According to the program described in Appendix 6, it is possible to generate the guidance report including the guidance content suitable for the personality of the target person receiving the health guidance, which thus can support the target person to continuously perform the activity based on the guidance content from the counselor.

What is claimed is:

1. An information processing device comprising:
a personality information acquisition unit configured to acquire personality information related to a personality of a target person receiving health guidance;
a health information acquisition unit configured to acquire health information including a result of a medical examination of the target person;
a goal setting unit configured to set a weight loss goal of the target person based on the health information;
a storage unit configured to store a trained model in advance, the trained model being configured to output, among a plurality of advice candidates based on the personality information, an advice candidate expected to be valid to achieve the weight loss goal as a guidance content for the target person, when the personality information and the weight loss goal are input; and
a report generation unit configured to input the personality information and the weight loss goal to the trained model and generate a guidance report including the guidance content output from the trained model.

2. The information processing device according to claim 1, wherein
the personality information acquisition unit acquires, as the personality information, whether the target person is motivated, a strength of will, and assertiveness of the target person.

3. The information processing device according to claim 1, wherein
the personality information acquisition unit acquires, as the personality information, a result of a medical interview sheet or a questionnaire used in a medical interview of the target person.

4. The information processing device according to claim 1, wherein
the personality information acquisition unit acquires, as the personality information, a result of a personality analysis performed on the target person by using a Johari window.

5. The information processing device according to claim 1, wherein
the health information includes, as the result of the medical examination, diagnostic results of a waist circumference, a neutral fat, a fasting blood glucose level, and a blood pressure of the target person, and
the goal setting unit sets the weight loss goal such that the diagnostic results of the waist circumference, the neutral fat, the fasting blood glucose level, and the blood pressure are less than diagnostic criterion values of metabolic syndrome.

6. A non-transitory computer-readable storage medium storing a program, the program being configured to cause a computer to execute:
acquiring personality information related to a personality of a target person receiving health guidance;
acquiring health information including a result of a medical examination of the target person;
setting a weight loss goal of the target person based on the health information;
reading a trained model from a storage unit, the trained model being configured to output, among a plurality of advice candidates based on the personality information, an advice candidate expected to be valid to achieve the weight loss goal as a guidance content for the target person, when the personality information and the weight loss goal are input; and
inputting the personality information and the weight loss goal to the trained model and generating a guidance report including the guidance content output from the trained model.
